SEQUENCE LISTING (3 sequences)

SEQ ID NO: 1 - protein, 9 aa, Artificial Sequence
  NDX-90 peptide
QQYNSYPLT

SEQ ID NO: 2 - DNA, 30 bp, Artificial Sequence
  NDX-90 peptide
tgtcagcaatataacagctatcttctcacg

SEQ ID NO: 3 - DNA, 30 bp, Artificial Sequence
  NDX-90 peptide
tgccaacagtacaatagttacccccttaca

(19) United States
(12) Patent Application Publication
(10) Pub. No.: US 2024/0016881 A1
(43) Pub. Date: Jan. 18, 2024

(54) Coronavirus Therapy

(71) Applicant: Arpi Rogers, London (GB)

(72) Inventor: Arpi Rogers, London (GB)

(21) Appl. No.: 18/029,124

(22) PCT Filed: Oct. 4, 2021
(86) PCT No.: PCT/GB2021/052554

§ 371 Date: Mar. 29, 2023

(30) Foreign Application Priority Data
Oct. 5, 2020 (GB) 2015755.8

(51) Int. Cl. A61K 38/08

(52) U.S. Cl. CPC A61K 38/08; A61K 48/00

(57) ABSTRACT

The invention provides a peptide, or a derivative or analogue thereof, or a nucleic acid encoding the peptide, or a derivative or analogue thereof, for use in treating, preventing or ameliorating a coronavirus infection or symptoms in an infected subject.

The present invention relates to coronavirus therapies, and particularly, although not exclusively, to novel compositions, therapies and methods for treating, preventing or ameliorating a coronavirus infection, or symptoms in an infected subject.

The severe acute respiratory syndrome coronavirus (SARS-CoV-2), causing 2019-nCoV or COVID-19 disease, is a virus belonging in the coronavirus (CoV) group of disease-causing pathogens that includes severe acute respiratory syndrome coronavirus (SARS) and Middle East respiratory syndrome-related coronavirus (MERS). Coronaviruses are usually restricted to their wild hosts (e.g. bats). However, both SARS and MERS, and more recently SARS-CoV-2, have all been transferred to humans, and this caused the SARS and MERS outbreaks of 2003, 2012, and 2019, respectively.

The emergence in late 2019 of the novel pathogenic SARS-CoV-2 in China, has led to a pandemic of severe proportions with consequent human suffering, both in terms of loss of life and also from social and economic disruption. Currently, treatment of confirmed cases is symptomatic with no targeted therapies available which could prevent or reduce the severity of the virus, including the number of hospitalisations and deaths. Developing a vaccine has its inherent challenges and even when successfully developed, the benefits could be transient or delayed, or possibly become inapplicable to future pandemics. Additionally, vaccinating individuals already diagnosed with COVID-19 disease is unlikely to have any disease-ameliorating effect due to the time required for protective immunity to develop post-vaccination.

The characteristics of the highly infectious SARS-CoV-2, demonstrate that the severity of the disease is associated with both age and chronic conditions, according to a report by the CDC COVID-19 Response Team (1). Preliminary data from 7162 COVID-19 infected patients, shows that 37.6% had one or more underlying health conditions. Additionally, it was reported that 78% of intensive care unit (ICU) patients with COVID-19 had at least one underlying health condition, including diabetes in 32%, cardiovascular disease in 29%, chronic lung disease in 21% and long-term kidney disease in 12% (1).

Underlying conditions such as Type 1 or Type 2 diabetes, hypertension, cardiovascular disease, kidney disease, and lung disease, have all been noted to contribute to the severity of SARS-CoV-2 infection. Thus far, however, the severity of COVID-19 has been considered to be a result of increased fragility caused by these co-morbidities.

It has been observed that not only Type 1 and Type 2 diabetes, but all of the conditions associated with diabetes, such as cardiovascular disease, kidney disease and lung disease, which are risk factors for severe COVID-19 infection and death, share two underlying metabolic features. These are ACE2 overexpression and glucoregulatory abnormalities, either overtly expressed as high blood glucose and glucagon as in Type 1 and Type 2 diabetes, or covertly expressed as in impaired glucose tolerance (IGT) or elevated fasting plasma glucose (FPG) in the absence of diabetes (2,3,4).

Glucoregulatory abnormalities have been observed in a number of conditions, including Type 1 and Type 2 diabetes, hypertension, cardiovascular disease, kidney disease, and lung disease. For example, abnormalities of glucose metabolism expressed as hyperinsulinemia, elevated FPG, and an abnormal response to an oral glucose tolerance test (OGTT), have all been shown to be important risk factors for cardiovascular disease (CVD (5, 6, 7). Additionally, Qiao et al., demonstrated that impaired glucose tolerance was an independent predictor for the incidence of chronic heart disease (CHD), premature death from chronic vascular disease (CVD), and death from all causes which was not confounded by the development of clinically diagnosed diabetes (8). Similarly, Sechi L A et al., reported that 321 hypertensive patients were found to have hyperinsulinemia, when compared to 92 matched normotensive subjects (9).

Hyperinsulinemia, also referred to as insulin resistance, is generally considered to be due to increased plasma glucose levels, because of a lack of accompanying measurements of glucagon levels. Based on OGTT studies, a distinction has been made between subjects who have normal fasting glucose levels and those who have raised fasting glucose levels, before glucose loading. The classification of subjects resulting from these tests is Normal Glucose Tolerance (NGT), Impaired Glucose Tolerance (IGT) or Impaired Fasting Glucose (IFG). The progression outcomes of IGT and IFG have been reported to be variable in both groups, with ˜25% progressing to diabetes and others remaining in their abnormal glycaemic state or reverting to NGT (10, 11).

Faerch K et al., examined glucagon and insulin measurements in their OGTT studies and reported that individuals with diabetes had 30% higher fasting glucagon levels and diminished early glucagon suppression, when compared with individuals with normal glucose tolerance. Insulin resistance, i.e. hyperinsulinemia, was also associated with higher fasting glucagon levels and diminished early glucagon suppression (12). Additionally, Itchikawa R et al., emphasised the role of glucagon hypersecretion in OGTT studies in prediabetes and mild type 2 diabetes subjects, who exhibited higher fasting plasma glucagon levels than subjects with normal glucose tolerance (13).

Reaven G M et al., documented hyperglucagonaemia, i.e. excess glucagon, in both obese and non-obese patients with non-insulin-dependent type 2 diabetes (NIDDM), compared to normal subjects (14). Plasma glucose, insulin, and free fatty acid (FFA) concentrations were all higher than normal in patients with NIDDM, whether obese or non-obese. Furthermore, day-long plasma glucagon concentrations were increased in both NIDDM groups, and a direct relationship was observed between the total plasma glucagon response and total plasma glucose levels (14).

Elevated glucagon levels are clearly the underlying cause of both hyperglycaemia and hyperinsulinemia, since glucagon causes endogenous glucose production by gluconeogenesis and glycogenolysis (15). Additionally, glucagon has a direct action on the β-cell via glucagon receptors to cause insulin secretion (16). Accordingly, glucagon levels are directly correlated to insulin resistance in normal subjects (17).

When considering glucagon levels and COVID-19, Yang J. K. et al. conducted a retrospective analysis of patients who suffered from SARS-CoV-2 infection (135 patients died, 385 patients survived) compared to 19 patients with non-SARS pneumonia. The results demonstrated that fasting plasma glucose (FPG) levels were significantly higher in the deceased group vs. survivors vs. non-SARS pneumonia (9.7+/−5.2 vs. 6.5+/−3.0 vs. 5.1+/−1.0 mmol/l p<0.01). In this study, survival analysis showed that elevated FPG levels were independently associated with an increased hazard ratio (HR) of mortality from COVID-19 (6).

It has been observed that Angiotensin Converting Enzyme 2 (ACE2), the target receptor for SARS-CoV-2, is significantly overexpressed in patients with such underlying disease conditions. For example, ACE2 protein expression has been shown to be enhanced in both Type 1 and Type 2 diabetes, including diabetic nephropathy, as observed in seven separate studies, covering a total of 961 subjects (5). Consequently, the higher the expression of this ACE2 receptor, the higher the viral load will be, resulting in a more severe infection.

There is, therefore, an urgent need to provide a novel, quick acting therapy, which can reduce ACE2 expression, to reduce or prevent coronavirus infection per se, and also treat or ameliorate the development of symptoms in a virally infected subject.

The inventor investigated the efficacy of a peptide, known as “NDX-90” (SEQ ID No: 1), on preventing, treating and/or ameliorating a coronavirus infection or coronavirus symptoms. This is based on the inventor's hypothesis that a reduction in glucagon levels in a subject, will result in a decreased expression of the ACE2 receptor of the subject's “host” cells. The inventor therefore investigated the effects of the NDX-90 peptide on glucagon levels and surprisingly demonstrated that the addition of NDX-90, reduces the stress-induced hypersecretion of glucagon from freshly isolated pancreatic islet cultures. In addition, the inventor observed that fasting plasma glucagon levels in subjects treated with NDX-90 were significantly reduced over a period of time, when compared with the placebo group.

Accordingly, the inventor believes that NDX-90 may be utilised as a therapeutic agent to prophylactically reduce or prevent a coronavirus infection from occurring in the first place, and also treat or ameliorate symptoms in a virally infected subject, by reducing ACE2 expression levels and therefore ACE2 receptor concentrations on host cells.

Thus, in a first aspect of the invention, there is provided a peptide comprising an amino acid sequence substantially as set out in SEQ ID No: 1, or a derivative or analogue thereof, or a nucleic acid encoding the peptide, or a derivative or analogue thereof, for use in treating, preventing or ameliorating a coronavirus infection or symptoms in an infected subject.

In a second aspect of the invention, there is provided a method of treating, preventing or ameliorating a coronavirus infection or symptoms in an infected subject, the method comprising, administering, or having administered, to a subject in need of such treatment, a therapeutically effective amount of a peptide comprising an amino acid sequence substantially as set out in SEQ ID No: 1, or a derivative or analogue thereof, or a nucleic acid encoding the peptide, or a derivative or analogue thereof.

As described in the examples, the inventor has surprisingly shown that the secretion of glucagon from isolated rat pancreatic islet cultures was significantly decreased in islets cultured in the presence of NDX-90 (i.e. SEQ ID No:1), compared to isolated islets cultured in culture medium alone. Even more surprisingly, the inventor observed that the glucagon secretion-normalising effect starts almost immediately upon NDX-90 peptide addition, achieving 32% reduction after 4 hours and close to normal levels after 24 hours. Additionally, as shown in Table 2, the inventor was surprised to observe that NDX-90 had a prolonged glucagon-normalising effect over several months, demonstrated by the decrease in fasting plasma glucagon levels. The inventor's work has therefore shown that the NDX-90 peptide reduces glucagon levels, which will subsequently reduce ACE2 expression, thereby demonstrating that it can be used not only in the treatment, but also prevention or prophylaxis, of a coronavirus infection.

Previously, the association of high ACE2 expression with underlying conditions, such as diabetes and hypertension, has been viewed as a reactive protective response, leading to increased anti-inflammatory outcomes that suppress the pro-inflammatory arm of the Renin Angiotensin System (RAS). This pro-inflammatory arm is characterised by Angiotensin Converting Enzyme (ACE) and Angiotensin Type 1 Receptor (AT1R), which are associated with fibrosis, insulin resistance, inflammation and vasoconstriction (18).

The balance between the pro-inflammatory and protective arms of the Renin Angiotensin System (RAS) is controlled by the activation of Adenine Mono Phosphate Kinase (AMPK) (19), which has been shown to be activated by high glucagon levels (20). The pharmacological activation of AMPK suppresses ACE and AT1R (18), and it has been widely recognised as beneficial in improving various diseases. For example, it has been demonstrated that activation of AMPK can improve cardio metabolic disease, protect heart function and delay heart failure, inhibit cardiac hypertrophy and cardiomyopathy, and treat atherosclerosis by improving endothelial function (21-25). As such, the activation of AMPK is generally viewed as beneficial to a number of disease states.

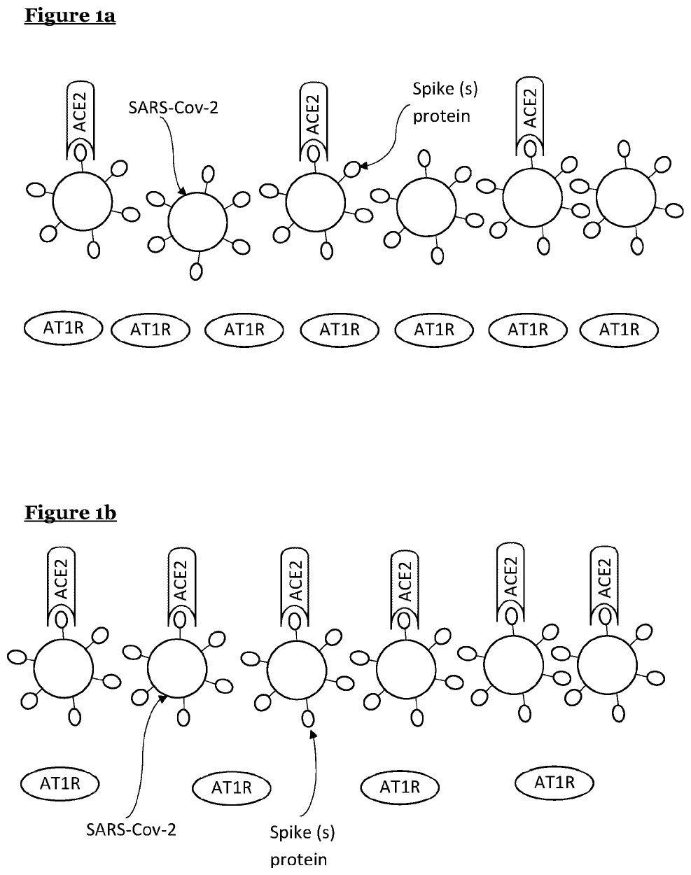

The inventor, however, has counterintuitively hypothesised that suppressing glucagon levels, thereby simultaneously reducing AMPK activation, would raise the levels of the pro-inflammatory ACE/AT1R arm of RAS and, as a result, lower the levels of the protective ACE2/Ang1-7 arm. This would redress the balance towards reduced ACE2 levels, thereby acting prophylactically and therapeutically against a coronavirus, such as SARS-CoV-2, which causes COVID-19. For example, as can be seen in FIG. 1a, normal glucagon levels are associated with higher AT1R expression (represented by seven AT1Rs) and lower ACE2 expression (represented by three ACEs), reducing the target for SARS-CoV-2 viral entry. As such, free virus particles that do not have a receptor to attach to will not successfully infect cells, and therefore, will die in the circulation due to the innate immune response. In contrast, as illustrated in FIG. 1b, high glucagon levels, which activate AMPK, and thus suppress AT1R expression (represented by four AT1Rs), resulting in increased ACE2 expression (represented by six ACEs) and increased targets for viral entry, leading to more severe viral infection of host cells.

On this basis, although not wishing to be bound by any hypothesis, the inventor believes that the most likely cause for increased mortality from coronavirus infection, in patients suffering from such underlying conditions, is due to their increased glucagon levels, resulting in the overexpression of ACE2, thereby leading to increased severity of the disease.

The inventor's hypothesis, however, is counterintuitive, and therefore non-obvious, for a number of reasons. Firstly, it has been recognised that ACE2 represents a protective mechanism against disease. Therefore, it would clearly be counterintuitive to consider lowering ACE2 levels in subjects suffering from such conditions. Additionally, the current understanding for the severity of COVID-19 and death observed in patients with cardiovascular, pulmonary and nephrotic conditions, is simply based on the accompanying fragility induced by these underlying conditions. Currently, no relationship has been observed between excess glucagon levels in such conditions and increased ACE2 expression. Furthermore, glucagon is a rarely studied parameter, even by experts of diseases associated with glucoregulatory failure, due to the prioritisation of glucose and insulin measurements, and as such, there are no diseases that are currently treated with glucagon-lowering drugs. Therefore, linking previously unconnected, multi-step processes that lead to raised levels of the pro-inflammatory arm of the RAS system (ACE/AT1R) to reduce the levels of the disease-protective arm (ACE2), thereby reducing ACE2 receptors to achieve protection against COVID-19 infection, is unexpected and unknown.

Additionally, one concept that has emerged, is that excess glucagon levels are caused by glucagon-enhancing autoantibodies, as a result of viral infections. T-cells multiply as a defensive immune response to viral infections, and the natural death of these T-cells is accompanied by the development of primary anti-T-cell receptor antibodies (anti-TCR). In certain individuals, this is followed by the development of a second generation of autoantibodies to the primary anti-TCR, i.e. anti-anti-T cell receptor antibodies (anti-anti-TCR). These anti-anti-TCR antibodies bind to α-cells in the human pancreas and cause uninterrupted secretion of glucagon. For example, it was demonstrated that anti-anti-TCR antibodies added to cultures of isolated pancreatic islets, raise glucagon levels by more than 50% within two days of culture compared to controls. This presence of anti-anti-TCR antibodies can explain the hyperglucagonaemia observed in Type 2 diabetes subjects.

It has been demonstrated that the acute phase of SARS-CoV-2 infection in humans results in T lymphocyte infection, through spike protein-mediated membrane fusion, and the infection of T-cells can cause marked lymphopenia of both CD4 and CD8 T-cells in over 80% of patients. Therefore, it appears that the destruction of T-cells due to infection with SARS-CoV-2, may further fuel the levels of anti-anti-TCR antibodies, tipping the balance of the glucagon/insulin ratio towards ketoacidosis. Production of these autoantibodies can be blocked by the NDX-90 peptide, which binds to the specific B-cell receptors and switches off the autoantibody producing B-cells. Therefore, suppression of such autoantibodies and normalisation of glucagon levels, is anticipated to ameliorate the severity of coronavirus infection, and the severity of viral infection symptoms and pathology.

Accordingly, preferably the peptide, derivative or analogue thereof, or the nucleic acid is capable of reducing and/or inhibiting the production of anti-anti-T cell receptor antibodies in a subject compared to the level of anti-anti-T cell receptor antibodies in an untreated subject.

It will be appreciated that a healthy subject has a physiologically “normal” concentration (or levels) of anti-anti-T cell receptor antibodies, and that a subject suffering from an underlying disease characterised by glucoregulatory abnormalities has an elevated concentration of these antibodies compared to the “normal” antibody concentration in a healthy subject. Accordingly, it is preferred that administration of the peptide, derivative or analogue thereof, or the nucleic acid, results in the concentration of anti-anti-T cell receptor antibodies being reduced by at least 70% towards the normal antibody concentration in a healthy subject. Preferably, administration of the peptide, derivative or analogue thereof, or the nucleic acid, results in the concentration of anti-anti-T cell receptor antibodies being reduced by at least 75%, 80%, 85%, 90% or 95% towards the normal antibody concentration in a healthy subject. Most preferably, administration of the peptide, derivative or analogue thereof, or the nucleic acid, results in the concentration of anti-anti-T cell receptor antibodies being reduced towards the normal antibody concentration in a healthy subject.

Alternatively, the concentration of anti-anti-T cell receptor antibodies are preferably maintained at the “normal” concentration in a healthy subject.

Preferably, the peptide, derivative or analogue thereof, or the nucleic acid is capable of reducing and/or inhibiting glucagon secretion in a subject compared to the level of glucagon secretion in an untreated subject.

It will be appreciated that a healthy subject has a physiologically “normal” concentration (or levels) of glucagon, and that a subject suffering from an underlying disease characterised by glucoregulatory abnormalities has an elevated concentration of glucagon compared to the “normal” glucagon concentration in a healthy subject. Accordingly, it is preferred that administration of the peptide, derivative or analogue thereof, or the nucleic acid, results in the concentration of glucagon being reduced by at least 70% towards the normal glucagon concentration in a healthy subject. Preferably, administration of the peptide, derivative or analogue thereof, or the nucleic acid, results in the concentration of glucagon being reduced by at least 75%, 80%, 85%, 90% or 95% towards the normal glucagon concentration in a healthy subject. Most preferably, administration of the peptide, derivative or analogue thereof, or the nucleic acid, results in the concentration of glucagon being reduced towards the normal glucagon concentration in a healthy subject.

Alternatively, the concentration of glucagon is preferably maintained at the “normal” glucagon concentration in a healthy subject.

It has been observed that isolated pancreatic islets secrete higher than normal levels of glucagon, due to the enzymatic digestion procedures which separate the intact islets from pancreatic tissue (Lee H B and Blaufox M D, J Nucl Med, 1985; 25:72-76). Preferably, therefore, glucagon secretion may be reduced and/or inhibited in the subject's pancreatic islets compared to the level of glucagon secretion in an untreated subject.

Glucagon secretion from pancreatic alpha-cells is accompanied by stoichiometric co-secretion of glutamate (26). Glutamate acts as a positive autocrine signal for glucagon release, and therefore, co-secretion of glutamate creates a continuous signal for further glucagon release (27), which will enhance the severity of coronavirus infection.

Therefore, preferably, the peptide, derivative or analogue thereof, or the nucleic acid is capable of reducing and/or inhibiting glutamate secretion in a subject compared to the level of glutamate secretion in an untreated subject.

It will be appreciated that a healthy subject has a physiologically “normal” concentration (or levels) of glutamate, and that a subject suffering from an underlying disease characterised by glucoregulatory abnormalities has an elevated concentration of glutamate compared to the “normal” glutamate concentration in a healthy subject. Accordingly, it is preferred that administration of the peptide, derivative or analogue thereof, or the nucleic acid, results in the concentration of glutamate being reduced by at least 70% towards the normal glutamate concentration in a healthy subject. Preferably, administration of the peptide, derivative or analogue thereof, or the nucleic acid, results in the concentration of glutamate being reduced by at least 75%, 80%, 85%, 90% or 95% towards the normal glutamate concentration in a healthy subject. Most preferably, administration of the peptide, derivative or analogue thereof, or the nucleic acid, results in the concentration of glutamate being reduced towards the normal glutamate concentration in a healthy subject.

Alternatively, the concentration of glutamate is preferably maintained at the “normal” glutamate concentration in a healthy subject.

Most preferably, the peptide, derivative or analogue thereof, or the nucleic acid is capable of reducing and/or inhibiting glucagon and glutamate secretion in a subject compared to the level of glucagon and glutamate secretion in an untreated subject.

Preferably, the peptide, derivative or analogue thereof, or the nucleic acid is capable of reducing and/or inhibiting ACE2 overexpression in a subject compared to the level of ACE2 expression in an untreated subject.

It will be appreciated that a healthy subject has a physiologically “normal” level of ACE2 expression, and that a subject suffering from an underlying disease characterised by glucoregulatory abnormalities has an elevated level of ACE2 expression compared to the “normal” ACE2 expression level in a healthy subject. Accordingly, it is preferred that administration of the peptide, derivative or analogue thereof, or the nucleic acid, results in the level of ACE2 expression being reduced by at least 70% towards the normal ACE2 expression level in a healthy subject. Preferably, administration of the peptide, derivative or analogue thereof, or the nucleic acid, results in the level of ACE2 expression being reduced by at least 75%, 80%, 85%, 90% or 95% towards the normal ACE2 expression level in a healthy subject. Most preferably, administration of the peptide, derivative or analogue thereof, or the nucleic acid, results in the ACE2 expression level being reduced towards the normal ACE2 expression level in a healthy subject.

Alternatively, the level of ACE2 expression is preferably maintained at the “normal” ACE2 expression level in a healthy subject.

Advantageously, NDX-90 targets glucagon secretion, and therefore, reduces ACE2 expression. Reducing the number of ACE2 receptors present on the surface of cells in the subject reduces the ability of the coronavirus to infect host cells, and is therefore prophylactic in nature. In contrast, a number of the current therapies used to treat diabetes and associated conditions, actually increase the number of ACE2 receptors. This includes SGLT2-inhibitors, pioglitazone, metformin, GLP-1 receptor agonists, and insulin, which have all been shown to upregulate ACE2 (28). As such, treatment with NDX-90 provides a significant advantage over previous therapies, which could increase the risk of infection by coronaviruses (e.g. SARS-CoV-2) and even worsen the prognosis of symptoms in a subject infected with a coronavirus (e.g. COVID-19).

Furthermore, in severe cases of coronavirus infection, mitochondrial damage occurs, resulting in the release of mitochondrial DNA (mtDNA), accompanied by the release of cardiolipin, which results in acute respiratory distress syndrome (ARDS), cytokine storm, and multi-organ failure. Additionally, high circulating mtDNA levels have been shown to be a potential early indicator for poor Covid-19 outcomes (29). As shown in Table 4, the inventor has demonstrated that NDX-90 has the capacity to bind to cardiolipin, and therefore, can prevent the shifting apart of the cardiolipin chains by holding the dimeric arms of cardiolipin together, thus improving the bilayer thickness of mitochondrial membranes and preventing the release of mtDNA.

Thus, preferably, the peptide, derivative or analogue thereof, or the nucleic acid is capable of binding to cardiolipin.

Even more preferably, the peptide, derivative or analogue thereof, or the nucleic acid is capable of reducing and/or inhibiting the release of mitochondrial DNA (mtDNA) from the mitochondria in a subject, compared to the level of mtDNA release from the mitochondria in an untreated subject.

It will be appreciated that a healthy subject has physiologically “normal” levels of mtDNA release, and that a subject suffering from a coronavirus infection has elevated levels of mtDNA release compared to the “normal” mtDNA release in a healthy subject. Accordingly, it is preferred that administration of the peptide, derivative or analogue thereof, or the nucleic acid, results in the levels of mtDNA release being reduced by at least 70% towards the normal levels of mtDNA release in a healthy subject. Preferably, administration of the peptide, derivative or analogue thereof, or the nucleic acid, results in the levels of mtDNA release being reduced by at least 75%, 80%, 85%, 90% or 95% towards the normal levels of mtDNA release in a healthy subject. Most preferably, administration of the peptide, derivative or analogue thereof, or the nucleic acid, results in the levels of mtDNA release being reduced towards the normal levels of mtDNA release in a healthy subject.

Alternatively, the levels of mtDNA release are preferably maintained at the “normal” levels of mtDNA release in a healthy subject.

The coronavirus may be any virus belonging to the coronavirus group of disease-causing pathogens, which targets the Angiotensin Converting Enzyme 2 (ACE2) receptor on a host cell for infection thereof. Accordingly, preferably the peptide comprising SEQ ID No: 1, or a derivative or analogue thereof, or the nucleic acid encoding the peptide, or a derivative or analogue thereof, prevents a coronavirus infection, or treats or ameliorates symptoms in a subject infected with any coronavirus which targets, and infects a host cell, via the ACE2 receptors.

Preferably, the coronavirus is selected from MERS, SARS-CoV-1 and SARS-CoV-2. Most preferably, however, the coronavirus is SARS-CoV-2 and any future strain with ACE2 target preference. It will be appreciated that SARS-CoV-2 is the causative agent of COVID-19.

The characteristics of the highly infectious SARS-CoV-2, demonstrate that the severity of the disease is associated with underlying conditions, such as Type 1 or Type 2 diabetes, hypertension, cardiovascular disease, kidney disease, and lung disease. In particular, these underlying conditions and any other conditions characterised by glucoregulatory abnormalities.

Hence, in one embodiment, the peptide, derivative or analogue thereof, or the nucleic acid is capable of treating, preventing or ameliorating a coronavirus infection or symptoms in an infected subject who suffers from an underlying disease. In particular, the underlying disease may be characterised by a glucoregulatory abnormality. Preferably, the disease is associated with impaired glucose tolerance or elevated fasting plasma glucose. Even more preferably, the disease is associated with high glucagon levels (i.e. hyperglucagonemia) or insulin resistance (i.e. hyperinsulinemia). Preferably, the subject is glucose-intolerant.

Preferably, the disease is selected from a group consisting of: diabetes; Type 1 diabetes; Type 2 diabetes; hypertension; cardiovascular disease; kidney disease; and lung disease. The subject may be a coronavirus-infected diabetic patient, preferably a SARS-CoV-2-infected diabetic patient.

In an alternative embodiment, the subject does not suffer from an underlying disease. In other words, the subject is a healthy individual. Accordingly, the subject may be a non-diabetic patient. The subject may be a coronavirus-infected non-diabetic patient, preferably a SARS-CoV-2-infected non-diabetic patient.

Preferably, the peptide, derivative or analogue thereof or the nucleic acid is capable of treating, preventing or ameliorating a coronavirus infection or symptoms in an infected subject who is in their 20's, 30's, 40's, 50's, 60's, 70's, 80's or 90's.

As described in the Examples, the inventor has shown that the NDX-90 peptide (SEQ ID No: 1) reduces glucagon secretion, thereby indicating that this peptide can be used in all conditions with underlying hyperglucagonemia as effective therapy for hyperglucagonemia and/or hyperinsulinemia.

Thus, in another aspect of the invention, there is provided a peptide comprising an amino acid sequence substantially as set out in SEQ ID No: 1, or a derivative or analogue thereof, or a nucleic acid encoding the peptide, or a derivative or analogue thereof, for use in treating, preventing or ameliorating hyperglucagonemia, hyperinsulinemia and/or a condition characterized by high or excessive glutamate

In a further aspect of the invention, there is provided a method of treating, preventing or ameliorating hyperglucagonemia, hyperinsulinemia and/or a condition characterized by high or excessive glutamate in a subject, the method comprising, administering, or having administered, to a subject in need of such treatment, a therapeutically effective amount of a peptide comprising an amino acid sequence substantially as set out in SEQ ID No: 1, or a derivative or analogue thereof, or a nucleic acid encoding the peptide, or a derivative or analogue thereof.

Preferably, the peptide, derivative or analogue thereof, or the nucleic acid is as defined herein.

For example, a condition characterized by hyperglucagonemia may be Type 2 or Type 1 diabetes, which may accompanied initially by hyperinsulinemia due to high glucagon. As the diseases progress, co-secreted high or excessive glutamate blocks mature insulin secretion by ˜/>50% in Type 2 and ˜/>95% in Type 1 diabetes. Glutamate is transported to the secretory granules in β-cells where it contributes to the maturation of insulin. Glutamate causes acidification in the secretory granules which stimulates conversion of pro-insulin to insulin. Excessive glutamate promotes faster secretion thus favoring pro-insulin secretion and insufficient time for acidification/maturation of pro-insulin to insulin. High pro-insulin and microsecretion of mature insulin is a characteristic of Type 1 diabetes and also present to a lesser degree in Type 2 diabetes. Diseases with underlying hyperglucagonemia and hyperinsulinemia also include hypertension, chronic heart disease, cardiovascular disease, kidney disease, chronic lung disease, obesity and cancers. Impaired Glucose Tolerance (IGT), Impaired Fasting Glucose/Glucagon (IFG) are also underlying conditions that can be treated prophylactically to prevent progression to serious disease.

Diseases of high plasma or blood glutamate levels include Alzheimer's disease, chronic schizophrenia, Major Depressive Disorder (MDD), Autism Spectrum Disorder, Multiple Sclerosis, Parkinson's disease and neuro-muscular degenerative disorders.

Preferably, the peptide, derivative or analogue thereof, or the nucleic acid may be used in the effective prophylaxis, amelioration, or treatment of a coronavirus infection or symptoms in an infected subject, preferably by reducing and/or inhibiting ACE2 expression in a subject compared to the level of ACE2 expression in an untreated subject.

In one embodiment, the protein sequence of the peptide, referred to as “NDX-90” is 9 amino acids in length, and is provided herein as SEQ ID No: 1 as follows:

[SEQ ID No: 1]
QQYNSYPLT

In one embodiment, the peptide is linked together with a second peptide to form a dimer. Preferably, the second peptide also comprises an amino acid sequence substantially as set out in SEQ ID No: 1, or a derivative or analogue thereof. Preferably, the peptides are linked by a cysteine residue at the N-terminal of each peptide.

The peptide may be reduced in size by the removal of amino acids. The reduction of amino acids may be achieved by removal of residues from the C- and/or N-terminal of the peptide, or may be achieved by deletion of one or more amino acids from within the core of the peptide.

The term “derivative or analogue thereof” can mean a peptide within which amino acid residues are replaced by residues (whether natural amino acids, non-natural amino acids or amino acid mimics) with similar side chains or peptide backbone properties. Additionally, the terminals of such peptides may be protected by N- and/or C-terminal protecting groups with similar properties to acetyl or amide groups.

Derivatives and analogues of peptide according to the invention may also include those that increase the peptide's half-life in vivo. For example, a derivative or analogue of the peptide of the invention may include peptoid and retropeptoid derivatives of the peptides, peptide-peptoid hybrids and D-amino acid derivatives of the peptides.

Peptoids, or poly-N-substituted glycines, are a class of peptidomimetics whose side chains are appended to the nitrogen atom of the peptide backbone, rather than to the alpha-carbon, as they are in amino acids. Peptoid derivatives of the peptides of the invention may be readily designed from knowledge of the structure of the peptide. Retropeptoids (in which all amino acids are replaced by peptoid residues in reversed order) are also suitable derivatives in accordance with the invention. A retropeptoid is expected to bind in the opposite direction in the ligand-binding groove, as compared to a peptide or peptoid-peptide hybrid containing one peptoid residue. As a result, the side chains of the peptoid residues are able point in the same direction as the side chains in the original peptide.

Preferred nucleic acid molecules according to the invention may include:—

[SEQ ID No: 2]
TGTCAGCAATATAACAGCTATCTTCTCACG
[SEQ ID No: 3]
TGCCAACAGTACAATAGTTACCCCCTTACA

Preferably, therefore, the nucleic acid molecule comprises a nucleotide sequence substantially as set out in any one of SEQ ID No: 2 or 3, or a variant or fragment thereof.

The nucleic acid molecule may be an isolated or purified nucleic acid sequence. The nucleic acid sequence may be a DNA sequence. The nucleic acid molecule may comprise synthetic DNA. The nucleic acid molecule may comprise cDNA. The nucleic acid may be operably linked to a heterologous promoter. The nucleic acid sequence encoding the peptide, derivative or analogue thereof, may be incorporated into a genetic construct for use in gene therapy or for cloning purposes.

In one preferred embodiment, therefore, the nucleic acid molecule encoding the peptide, derivative or analogue thereof is a genetic construct. More preferably, the nucleic acid molecule or genetic construct is provided in a recombinant vector.

Genetic constructs of the invention may be in the form of an expression cassette, which may be suitable for expression of the encoded peptide, derivative or analogue thereof, in a host cell. The genetic construct may be introduced into a host cell without it being incorporated in a vector. For instance, the genetic construct, which may be a nucleic acid molecule, may be incorporated within a liposome or a virus particle. Alternatively, a purified nucleic acid molecule (e.g. histone-free DNA, or naked DNA) may be inserted directly into a host cell by suitable means, e.g. direct endocytotic uptake. For cloning purposes, the genetic construct may be introduced directly into cells of a host subject (e.g. a bacterial, eukaryotic or animal cell) by transfection, infection, electroporation, microinjection, cell fusion, protoplast fusion or ballistic bombardment. Alternatively, genetic constructs of the invention may be introduced directly into a host cell using a particle gun. Alternatively, the genetic construct may be harboured within a recombinant vector, for expression in a suitable host cell. For administration to the subject being treated, the construct may be contained in a phage delivery system, such as AAV.

The recombinant vector may be a plasmid, cosmid or phage. Such recombinant vectors are useful for transforming host cells with the genetic construct, and for replicating the expression cassette therein. The skilled technician will appreciate that genetic constructs of the invention may be combined with many types of backbone vector for expression purposes. Recombinant vectors may include a variety of other functional elements including a suitable promoter to initiate gene expression. For instance, the recombinant vector may be designed such that it autonomously replicates in the cytosol of the host cell. In this case, elements, which induce or regulate DNA replication, may be required in the recombinant vector. Alternatively, the recombinant vector may be designed such that it integrates into the genome of a host cell. In this case, DNA sequences which favour targeted integration (e.g. by homologous recombination) are envisaged.

To facilitate cloning, the recombinant vector may also comprise DNA coding for a gene that may be used as a selectable marker in the cloning process, i.e. to enable selection of cells that have been transfected or transformed, and to enable the selection of cells harbouring vectors incorporating heterologous DNA. Alternatively, the selectable marker gene may be in a different vector to be used simultaneously with vector containing the gene of interest. The vector may also comprise DNA involved with regulating expression of the coding sequence, or for targeting the expressed polypeptide to a certain part of the host cell.

It will be appreciated that the peptide, derivative or analogue thereof according to the invention may be used in a medicament, which may be used as a monotherapy (i.e. use of the peptide, derivative or analogue thereof, or the nucleic acid, alone), for treating, preventing or ameliorating a coronavirus infection or symptoms in an infected subject. Alternatively, the peptide, derivative or analogue thereof or nucleic acid according to the invention may be used as an adjunct to, or in combination with, known therapies for treating, preventing or ameliorating a coronavirus infection or symptoms in an infected subject. For example, the subject may be additionally treated with Remdesivir®, aspirin, dexamethasone and/or vitamin D.

The peptide or nucleic acid according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the peptide according to the invention may be used in a number of ways. For instance, oral administration may be required, in which case the peptide may be contained within a composition that may, for example, be ingested orally in the form of a tablet, capsule or liquid. As shown in Tables 5 and 6, the peptide of the invention (NDX-90) demonstrated efficient uptake by buccal tissue cells, showing that oral administration is an effective mode of delivering NDX-90 to the subject.

Preferably, therefore, the peptide of the invention is administered orally, and most preferably, sub-lingual administration. The peptide may be formulated with a mucosal adhesive, preferably for adhering to the buccal mucosa.

The peptide of the invention may be formulated with a cell membrane permeability enhancer, penetration enhancer and/or absorption enhancer.

The peptide according to the invention may also be incorporated within a slow- or delayed-release device. Such devices may, for example, be inserted on or under the skin, and the medicament may be released over weeks or even months. The device may be located adjacent the treatment site. Such devices may be particularly advantageous when long-term treatment with peptides used according to the invention is required and which would normally require frequent administration (e.g. daily injection).

In a preferred embodiment, medicaments according to the invention may be administered to a subject by injection into the blood stream or directly into a site requiring treatment. For example, the medicament may be injected close to, or at least adjacent to the pancreatic islets. Injections may be intravenous (bolus or infusion), intramuscular (bolus or infusion), subcutaneous (bolus or infusion), or intradermal (bolus or infusion).

It will be appreciated that the amount of the peptide that is required is determined by its biological activity and bioavailability, which in turn depends on the mode of administration, the physiochemical properties of the peptide and whether it is being used as a monotherapy or in a combined therapy. The frequency of administration will also be influenced by the half-life of the peptide within or on the subject being treated. Optimal dosages to be administered may be determined by those skilled in the art, and will vary with the particular peptide in use, the strength of the pharmaceutical composition, and the mode of administration. Additional factors depending on the particular subject being treated will result in a need to adjust dosages, including subject age, weight, gender, diet, and time of administration.

Optimal dosages may be determined depending on the severity of the coronavirus infection. The peptide may be administered once or twice a day for infected subjects who are hospitalised. Infected subjects who are not hospitalised may require less frequent administration of the peptide, such as once or twice a week, and/or lower doses than hospitalised subjects. Alternatively, the peptide may be administered even less frequently when being used as a prophylactic treatment. For example, weekly or monthly administration of the peptide may be required for uninfected subjects with pre-existing disease conditions which contribute to the severity of coronavirus infection. Alternatively, the peptide may be administered every month, 3-months, or 6-months, to uninfected subjects with normal or impaired glucose tolerance.

Generally, a daily dose of between 0.001 μg/kg of body weight and 10 mg/kg of body weight, or between 0.01 μg/kg of body weight and 1 mg/kg of body weight, of the peptide according to the invention may be used for treating, preventing or ameliorating a coronavirus infection or symptoms in an infected subject.

The peptide may be administered before, during or after onset of symptoms associated with a coronavirus infection. Daily doses may be given as a single administration (e.g. a single daily application). Alternatively, the peptide may require administration twice or more times during a day. As an example, the peptide may be administered as two (or more) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of the peptide according to the invention to a patient without the need to administer repeated doses.

Known procedures, such as those conventionally employed by the pharmaceutical industry (e.g. in vivo experimentation, clinical trials, etc.), may be used to form specific formulations of the peptide according to the invention and precise therapeutic regimes (such as daily doses of the agents and the frequency of administration). The inventor believes that they are the first to suggest a coronavirus treatment composition, based on the use of the peptide of the invention.

Hence, in a third aspect of the invention, there is provided a coronavirus infection prevention, treatment or amelioration pharmaceutical composition comprising a therapeutically effective amount of the peptide comprising an amino acid sequence substantially as set out in SEQ ID No: 1, or a derivative or analogue thereof, or a nucleic acid encoding the peptide, or a derivative or analogue thereof, and a pharmaceutically acceptable vehicle.

The invention also provides in a fourth aspect, a process for making the coronavirus infection prevention, treatment or amelioration pharmaceutical composition according to the third aspect, the process comprising combining a therapeutically effective amount of the peptide comprising an amino acid sequence substantially as set out in SEQ ID No: 1, or a derivative or analogue thereof, or a nucleic acid encoding the peptide, or a derivative or analogue thereof, with a pharmaceutically acceptable vehicle.

A “subject” may be a vertebrate, mammal, or domestic animal. Hence, medicaments according to the invention may be used to treat any mammal, for example livestock (e.g. a horse), pets, or may be used in other veterinary applications. Most preferably, however, the subject is a human being.

A “therapeutically effective amount” of peptide is any amount which, when administered to a subject, is the amount of active agent that is needed to treat, ameliorate, or prevent a coronavirus infection, or produce the desired effect. The peptide, derivative or analogue thereof may be used as an adjuvant for the prevention or treatment of a coronavirus infection. This means that lower doses of other prophylactic or therapeutic treatments would be required.

For example, the therapeutically effective amount of peptide used may be from about 0.001 mg to about 800 mg, and preferably from about 0.01 mg to about 500 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, coatings, or tablet-disintegrating agents. The vehicle may also be an encapsulating material. In powders, the vehicle is a finely divided solid that is in admixture with the finely divided active agents according to the invention. In tablets, the active agent (i.e. the peptide or nucleic acid) may be mixed with a vehicle having the necessary compression properties in suitable proportions and compacted in the shape and size desired. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention (i.e. the peptide or nucleic acid) may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The peptide may be prepared as a sterile solid composition that may be dissolved or suspended at the time of administration using sterile water, saline, or other appropriate sterile injectable medium.

The peptide and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The peptide used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Preferably, orally administrable formulations do not dissolve in the stomach, but preferentially dissolve in the duodenum. Orally administrable formulations may be enterically-coated, for example enteric-coated tablets or capsules. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID No:1-3, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence, which hybridizes to DNA sequences or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar peptide may differ by at least 1, 2, 3, 4 or 5 amino acids from the sequences shown in SEQ ID No: 1-3.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1a shows the effects of normal glucagon levels on the severity of infection with SARS-CoV-2. Normal glucagon levels are associated with higher expression of AT1R (represented by seven AT1Rs), and lower expression of ACE2 receptors (represented by three ACE2s). Consequently, there are fewer targets for SARS-CoV-2 viral entry, and free virus particles that do not have a receptor to attach to will die in the circulation.

FIG. 1b shows the effects of elevated glucagon levels on the severity of infection with SARS-CoV-2. High glucagon levels activate AMPK, which suppresses AT1R expression (i.e. fewer than there are in FIG. 1a as is represented by four AT1Rs) and increases ACE2 expression (i.e. more than there are in FIG. 1a as is represented by six ACE2s). This provides increased targets for viral entry and thus more severe infection with SARS-CoV-2.

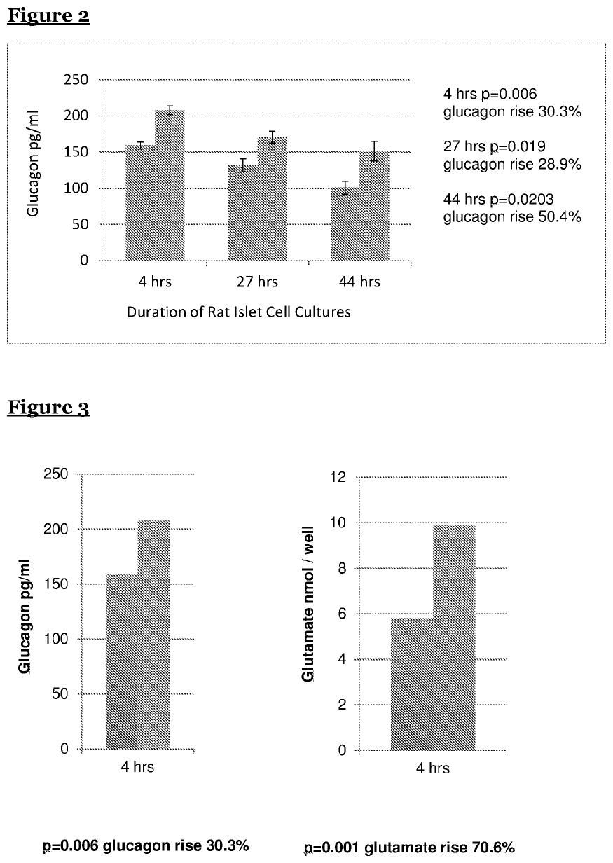

FIG. 2 illustrates glucagon secretion in rat islet cells cultured in the presence of cell culture medium alone (blue), and cell culture medium and anti-anti-TCR antibodies (green).

FIG. 3 illustrates the simultaneous increase of glucagon (green bar) and glutamate (orange bar) secretion from rat islet cells when cultured in the presence of anti-anti-TCR antibodies, compared to cells cultured in cell culture medium alone (blue bars).

### EXAMPLES

The inventor set out to test the hypothesis that the NDX-90 peptide can reduce glucagon levels in a subject, thereby decreasing the expression of the ACE2 receptor in the subjects “host” cells, and therefore, provide a novel therapy for treating, preventing or ameliorating a coronavirus infection, or symptoms in an infected subject.

Materials and Methods

Measuring Glucagon Levels Secreted from Isolated Rat Islet Cultures

Isolated islets were suspended in RPMI 1640 containing 11 mmol/L (200 mg/dL) glucose and 10% FCS. The glucose concentration simulates a diabetic environment. NDX-90 was dissolved in the culture medium and added to the appropriate wells at a final concentration of 1 μg/ml. After the specified incubation times, samples were removed for glucagon measurements using Quantikine ELISA Immunoassay (R&D Systems).

Cell Permeation Experiments

Buccal tissue culture inserts were transferred from the original tissue culture 24-well plates (maintained at 4° C. in sealed bags containing 5% CO₂) under sterile conditions into wells of a fresh 24-well plate containing 1.0 ml serum free medium pre-warmed to 37° C. The plate was incubated in a humidified incubator at 37° C. in a 5% CO₂ atmosphere for one hour as a pre-equilibration procedure prior to dosing. After removal from the incubator, any culture medium over the surface of the tissue was carefully removed and replaced with 40 μL of appropriately diluted peptide solution in serum free culture medium. The plate was incubated for further 30 minutes at 37° C. in a 5% CO₂ atmosphere. The plate was removed from the incubator and supernatant samples were taken and analysed by HPLC. The main peak areas of the supernatants were compared to those of the pre-incubation stock samples.

### Example 1—Effect of NDX-90 Peptide on Glucagon Levels Secreted from Isolated Rat Islet Cultures

The inventor measured glucagon levels secreted from isolated rat islet cultures, to determine whether NDX-90 was capable of reducing glucagon levels.

TABLE 1
Glucagon levels secreted from isolated rat islet cultures in culture
medium alone compared to cultures in presence of NDX-90 peptide

| | Glucagon level | | | |
|---|---|---|---|---|
| Time | (pg/ml) | Glucagon level (pg/ml) | Difference | Percentage |
| (hours) | Medium alone | Medium plus NDX-90 | (pg/ml) | reduction |
| 4 | 622.3 ± 5.5 | 419.3 ± 2.5 | 203.0 | 32.4% |
| 24 | 252.5 ± 6.3 | 149.6 ± 6.7 | 102.9 | 40.9% |
| 48 | 60.9 ± 3.8 | 63.4 ± 1.3 | | 0% |

As shown in Table 1, the inventor surprisingly discovered that the hypersecretion normalising effect starts almost immediately upon NDX-90 peptide addition. Following 4 hours, a 32% reduction of glucagon secretion was observed, and following 24 hours, glucagon secretion was reduced by 40.9%, reaching close to normal levels.

Thus, the data clearly and surprisingly demonstrate that the addition of NDX-90 peptide to isolated pancreatic islet cultures, reduces this stress-induced hypersecretion of glucagon from freshly isolated islets, very early after addition of the peptide.

### Example 2—Effect of NDX-90 Peptide Treatment on Fasting Plasma Glucagon

The inventor then measured the effect of NDX-90 peptide treatment over several months on fasting plasma glucagon levels in subjects with Type 2 diabetes.

TABLE 2
Effect of NDX-Peptide treatment on Fasting Plasma Glucagon.
Comparison of mean baseline fasting plasma glucagon on day
1 with mean of measurements on days 43, 99 and 113.

| | Baseline ± | Treated ± | |
|---|---|---|---|
| Treatment | sd pg/ml | sd pg/ml | Difference |
| NDX-90 Peptide | 103 ± 38.3 | 93.47 ± 32.3 | P = 0.009 |
| (n = 23) | | | 9.61 pg/ml |
| | | | (2.76 pmol/L) |
| Placebo group | 95.57 ± 20.2 | 94.28 ± 21.7 | P = 0.843 |
| (n = 8) | | | 1.29 pg/ml |
| | | | (0.37 pmol/L) |

As shown in Table 2, the mean fasting plasma glucagon level was significantly reduced (9.61 pg/ml, P=0.009) in subjects who received NDX-90 peptide, as compared to the placebo group (1.29 pg/ml, P=0.0843). These results demonstrate that the NDX-90 peptide has a prolonged glucagon normalizing effect over several months.

Additionally, the mean fasting glucagon concentrations in Type 2 diabetes were reported to be 3.5 pmol/L (12.2 pg/ml) higher than nondiabetic control subjects (P=0.012) (Menge B, Gruber L et al., Diabetes, 2011; 60:2160-2168). Therefore, the mean 9.61 pg/ml improvement in fasting glucagon levels in Type 2 diabetes patients shown in Table 2, represents a 78.8% improvement towards normality.

The importance of normalising glucagon levels provides benefits for the prevention or therapy of coronavirus infection that go beyond normalising glucagon secretion alone. Glucagon secretion from pancreatic alpha-cells is accompanied by stoichiometric co-secretion of glutamate (26). Firstly, glutamate acts as a positive autocrine signal for glucagon release, and therefore, co-secretion of glutamate creates a continuous signal for further glucagon release (27), which will enhance the severity of coronavirus infection unless the cycle is interrupted. Secondly, raised glutamate levels (also referred to as glutamic acid), have been shown to correlate with increased severity of Covid-19 disease (301. Additionally, glutamate levels in plasma of 132 Type II diabetics have been shown to be significantly higher than in 137 control subjects (P<0.01) (31).

Therefore, hypersecretion of glucagon that is co-secreted with glutamate, exerts a double impact on the severity of Covid-19 disease with associated metabolic dysregulation and neurological complications. In particular, glutamate is an important neurotransmitter that when unbalanced, can cause neurological abnormalities. Furthermore, it has been demonstrated that excess glutamate and glutamate transporters are associated with Sars-Cov-2 infection and disease severity. Additionally, high glutamate levels in Covid-19 infected patients have been implicated in disease severity as glutamate is utilised by the virus for its replication (32).

As previously stated, excess glucagon levels are caused by glucagon-enhancing autoantibodies (anti-anti-TCR antibodies), as a result of viral infections. As illustrated in Table 3 and FIG. 2, the inventor has demonstrated that glucagon secretion in rat islet cell cultures is significantly increased when cultured in the presence of anti-anti-TCR antibodies, compared to culture medium alone.

TABLE 3
Raised glucagon secretion in rat islet cultures
in the presence of anti-anti-TCR antibodies.

| | Mean glucagon concentration pg/ml | | | |
|---|---|---|---|---|
| | | Medium + | | |
| Time (hours) | Medium | anti-anti- TCR | p value | % rise |
| 4 | 159.4 ± 9.6 | 207.7 ± 12.2 | 0.0057 | 30.3 |
| 27 | 131.8 ± 17.9 | 170.5 ± 16.3 | 0.019 | 28.9 |
| 44 | 100.8 ± 17.9 | 151.6 ± 27.1 | 0.020 | 50.4 |

Furthermore, rat islet cells cultured in the presence of culture medium alone, or with additional anti-anti-TCR antibodies were examined for co-secretion of glucagon and glutamate. There was a highly significant increase in both glucagon and glutamate secretion within 4 hours of culture in the presence of anti-anti-TCR antibodies compared to control cells cultured in medium alone. Specifically, glutamate levels co-secreted from the same secretory granules as glucagon, were measured from the same culture supernatants from which glucagon levels were measured, and were significantly higher than glucagon levels (see FIG. 3).

Following infection and viral entry in the central nervous system (CNS), ACE2 receptors mediate the development of neurotoxicity, neuroinflammation and neurodegeneration via viral entrance and replication. Excessive glutamate accumulation causes progression of inflammatory neurodegeneration by increasing oxidative stress (33). Neurological symptoms occur frequently in hospitalised patients with Covid-19. Brain involvement may occur via several routes including the blood stream, infected neurons, olfactory nerves, ocular epithelium and impaired blood-brain barrier. Therefore, reduction of glutamate excitotoxicity by reducing glutamate levels could prevent or ameliorate neurological symptoms in coronavirus infection.

In two studies of hospitalised Covid-19 patients of 214 and 8.41 cases, neurological manifestations were observed in 36.4% and 57.4% of the cases respectively (34). Furthermore, it has been reported that in cross-sectional studies, high glutamate levels were associated with higher body mass index (BMI), high blood pressure and insulin resistance including diabetes (35). In a clinical trial of 980 participants, men and women of 55-80 years old, who had either type-2 diabetes or at least 3 cardiovascular disease (CVD) risk factors, were treated by Mediterranean diet compared to normal control diet. Zeng et al., reported that baseline glutamate was associated with 43% and 81% increased risk of composite CVD or stroke alone respectively. The Mediterranean diet followed up over a period of several years, lowered CVD risk by 37% compared to the control diet. As the conditions associated with high glutamate, i.e. higher BMI, high blood pressure and insulin resistance, are also associated with severity of Covid-19 infection, reducing glucagon and co-secreted glutamate levels within a few hours of administration of NDX-90, will also be highly effective in the prevention or amelioration of Covid-19 disease, as well as the underlying conditions which predispose to the severity of Covid-19 outcomes.

### Example 3—Ability of NDX-90 Peptide to Bind Cardiolipin

Cardiolipin has a significant role in mitochondrial bioenergetics due to its almost exclusive association with mitochondrial membranes, designed to produce ATP through the electrochemical gradient generated by the electron transport chain. Mitochondrial respiratory chain complexes, referred to as respiratory supercomplexes, are involved in oxidative phosphorylation and maintain their structural integrity and activity via the unique dimeric cross-linked structure of cardiolipin. Cardiolipin contains unsaturated fatty acyl chains, which are oxidisable targets. Peroxidation of cardiolipin alters its structural integrity and is considered to lead to mitochondrial dysfunction associated with age and various pathophysiological conditions, including diabetes and cardiovascular disorders (36).

Detailed analysis has revealed that oxidation of cardiolipin leads to a conformational change in the backbone/head group and in the chain regions of the oxidised cardiolipin molecules. The oxidised groups have been observed to shift apart, increasing the area per lipid chain and decreasing the bilayer thickness, thus altering the functionality of the inner mitochondrial membrane (37).

TABLE 4
Optical density readings in ELISA assays showing
binding of NDX-90 peptide to cardiolipin

| | |
|---|---|
| Cardiolipin** | 0.254 ± 0.012 |
| Alcohol** | 0.114 ± 0.021 |
| Ratio | 2.23 |
| Test/Control | |

*The microtitre plate was coated with either cardiolipin in ethyl alcohol or ethyl alcohol and tested against NDX-90.

Each mean and standard deviation is for three observations.

As shown in Table 4, NDX-90 (which is a dimer) has the capacity to bind to cardiolipin (also a dimer). The inventor believes that NDX-90 may function by holding the dimeric arms of cardiolipin together, thus preventing them shifting apart and maintaining the cardiolipin-dependent bioenergetic processes in the mitochondrial inner membrane. By binding to cardiolipin, NDX-90 has a therapeutic effect in subjects suffering from diabetes or other conditions characterised by insulin resistance and high glucagon levels, which are associated with mitochondrial dysfunction. Therefore, the surprising cell-penetrating mechanistic capacity of NDX-90 described above is believed to contribute to the therapeutic function of NDX-90. In severe cases of Covid-19 infection, mitochondrial damage occurs, resulting in the release of mitochondrial DNA (MT-DNA), accompanied by the release of cardiolipin, which results in acute respiratory distress syndrome (ARDS), cytokine storm, and multi-organ failure. MT-DNAs are intrinsically inflammatory and highly elevated levels have been shown in the plasma of patients. High circulating MT-DNA levels have also been shown to be a potential early indicator for poor Covid-19 outcomes. Multivariate regression revealed that high circulating MT-DNA was an independent risk factor for ICU admission, intubation, vasopressor use or renal replacement therapy (29).

Cardiolipin is known to bind various mitochondrial proteins and thereby contribute to their integrity and function (37 Binding of NDX-90 dimer to the cardiolipin chains can prevent the shifting apart of the cardiolipin chains altered by oxidation, improve the bilayer thickness of mitochondrial membranes and prevent the release of MT-DNA.

### Example 4—Permeability of NDX-90 Peptide

The inventor next measured buccal tissue permeability of NDX-90 peptide, to determine its uptake. Table 5 summarises a preliminary permeability analysis of 3 peptides A, B and NDX-90, and Table 6 shows triplicate analyses for peptides A and NDX-90. Peptide A is a dimer of 17 amino acids per monomer, wherein the peptides are linked by a cysteine residue at the N-terminal of each peptide (i.e. peptide A is almost twice the size of NDX-90). Peptide B is a dimer of 8 amino acids per monomer, wherein the peptides are also linked by a cysteine residue at the N-terminal of each peptide.

TABLE 5
Buccal tissue permeability screening of 3 peptide solutions

| | Peak Area by HPLC | Peak Area by HPLC | |
|---|---|---|---|
| | of Peptides in | of Peptides in | |
| | solution | solution | Transmissibility |
| Peptide | Pre-overlay | Post-overlay | % |
| A | 5398 | 4439 | 17.7% |
| B | 2023 | 2396 | 0% |
| NDX-90 | 4142 | 243 | 94.1% |

TABLE 6
Buccal tissue permeability of peptides A and NDX-90 in triplicate cultures
Peak Area by HPLC of Peptide Solutions from Replicate(R) Cultures and
Transmissibility(T) %

| | Pre- | | | | | | |
|---|---|---|---|---|---|---|---|
| Peptide | overlay | R₁ | T % | R₂ | T % | R₃ | T % |
| A | 4840 | 2599 | 46.3% | 3208 | 33.7% | 3466 | 28.3% |
| NDX-90 | 4142 | 276 | 93.3% | 539 | 84.5% | 0 | 100 % |

As can be seen in Table 5, peptide B did not demonstrate any transmissibility across the tissue surface under the experimental conditions. However, NDX-90 clearly showed efficient uptake by the buccal tissue cells in the triplicate cultures. The comparison of NDX-90 with peptides A and B, demonstrates that its membrane transmissibility is an inherent property of the peptide, not a size effect, and it cannot be expected or predicted.

### Example 5—Ability of NDX-90 Peptide to Reduce ACE2 Levels

The inventor next measured the effect of NDX-90 peptide on soluble ACE2 levels in cultures of human islet cells from Type 2 diabetes tissue donors. Human pancreatic islet cells were cultured in 24-well tissue culture plates. Two series of cultures were set up. NDX-90 peptide solution was added to one series and an equivalent amount of culture medium was added to the second series. Supernatant samples were removed from the test and control cultures at 4, 24 and 48 hours after addition of the treatment. Supernatant samples were removed only once from each test and control well for each time point. The samples were tested for soluble ACE2 by the use of a commercial ELISA kit according to the manufacturer's instructions. Tests were performed in triplicate and mean Optical Density (OD) data are shown in Table 7 below.

TABLE 7
Soluble ACE-2 measurements in human donor Type 2 diabetes
islet cells cultured in the presence of NDX-90 peptide
compared to cultures in medium alone

| | OD Mean ± | OD Mean ± |
|---|---|---|
| Treatment | sd 4 hours | sd 24 hours |
| Control - None | 0.133 ± 0.011 | 0.128 ± 0.021 |
| Test - NDX-90 Peptide | 0.126 ± 0.013 | 0.120 ± 0.002 |
| % reduction soluble ACE2 | 5.3% | 6.25% |
| 100-(T/Cx100) | | |

The data demonstrate that at 4 hours after addition of peptide to the cultures, there was a 5.3% reduction in soluble ACE2 levels which was further reduced to 6.25% at 24 hours. No further reduction was observed with longer incubation time of 48 hours. This reflects the homeostatic control in physiological systems to maintain normality in the absence of overriding disease processes.

The impact of the reduction in soluble ACE2 due to NDX-90 outlined above, is apparent in view of the publication by Kragstrup T W (38). In this publication (FIG. 2), plasma ACE2 levels from 305 hospitalised Covid-19 patients were expressed as relative protein values using Normalised Protein eXpression (NPX), on a scale of 0 to 8 with all but one datum point within 0 to 6. Patients were categorised into 5 groups based on disease severity from death within 28 days (group 1) to less severe groups 2-5, with and without oxygen requirement. Median plasma ACE2 levels at day 0 of hospitalisation were determined for each group. When the range of plasma ACE2 levels within the range of 0 to 6 is subdivided from 0 to 60, it becomes apparent that the difference between the medians of group 1 (death within 28 days) and groups 2-5 (survivors with or without oxygen) is only 4 units higher plasma ACE2 on the 0 to 60 range. Within this range with 60 units representing 100%, 4 units represent 6.6% (4/60×100).

This is remarkably close to the reduction of 6.25% in soluble ACE2 levels obtained from the human Type 2 diabetes islet cell cultures in the presence of NDX-90 compared to control cultures (see Table 7 above). Therefore, this clearly indicates that early NDX-90 treatment can, within 4 to 24 hours, reduce soluble ACE2 sufficiently to stop progression of Covid-19 infection and prevent death. Unlike in culture conditions demonstrated above, in vivo doses can be repeated according to the severity of the condition. This would prevent plasma ACE2 levels rising as seen in FIG. 3 of the Kragstrup T W publication on day 7 after hospitalisation (38). It is also a corollary, that treatment of individuals with underlying conditions of diabetes, hyperinsulinemia, hyperglucagonemia, cardiovascular disease and conditions associated with insulin resistance can benefit from NDX-90 treatment both for their particular condition and as therapy or prevention of Covid-19 disease.

Summary

SARS-coronavirus 2 has led to a pandemic of severe proportions with consequent human suffering, particularly in individuals with underlying conditions associated with impaired glucose tolerance. If an effective treatment could be given that reduces glucagon levels, thereby reducing ACE2 expression, it would have the potential to prophylactically prevent a coronavirus infection, or treat or ameliorate symptoms in an infected subject.

As described herein, the inventor administered the peptide, NDX-90, to isolated rat islet cultures, and observed a significant decrease in the secretion of glucagon compared to the control, very early after addition of the peptide (4 hours). The inventor also found that NDX-90 has a prolonged glucagon-normalizing effect over several months, effectively decreasing fasting plasma glucagon levels in subjects with Type 2 diabetes. This work has therefore shown that the NDX-90 peptide reduces glucagon secretion, thereby indicating that this peptide can be used in all conditions with underlying hyperglucagonemia as effective therapy for hyperglucagonemia and/or hyperinsulinemia, and the effective prophylaxis, amelioration, or treatment of a coronavirus infection and symptoms in an infected subject, by decreasing the expression of the ACE2 receptor.

Additionally, NDX-90 has demonstrated two further mechanisms of combatting Covid-19 infection and disease progression within a few hours of administration. Glucagon is co-secreted with glutamate from the same pancreatic islet alpha-cell secretory vesicles. This pancreatic source of glutamate constitutes the large part of excess glutamate in patients with insulin resistance, diabetes, cardiovascular manifestations, high blood pressure, obesity and other underlying conditions which predispose to severity of Covid-19 outcomes. Glutamate levels are elevated in Covid-19 infections and are the cause of neurological symptoms that aggravate the disease severity. Glutamate secretion is reduced simultaneously with glucagon secretion, as glutamate is co-secreted with glucagon from the same secretory granules.

The action of glutamate, however, is different from that of glucagon. Glutamate is utilized by the virus in its replication process, and therefore, high glutamate levels contribute to viral load and are associated with Covid-19 severity and poor survival outcomes. In two studies of hospitalised Covid-19 patients, neurological manifestations have been observed in 36.4% and 57.4% of the cases respectively. Therefore, reduction of glutamate levels by administration of NDX-90 would be both prophylactic and therapeutic for Covid-19 disease.

Furthermore, NDX-90 has the ability to bind to cardiolipin which is a unique phospholipid almost exclusively located in the inner mitochondrial membrane. In severe cases of Covid-19, mitochondrial damage occurs which results in the release of mitochondrial DNA (MT-DNA), accompanied by the release of cardiolipin which results in acute respiratory distress syndrome (ARDS) cytokine storm, and multi-organ failure. MT-DNAs are intrinsically inflammatory and highly elevated levels have been shown in the plasma of patients. High circulating MT-DNA levels have also been shown to be a potential early indicator for poor Covid-19 outcomes. Therefore, binding of NDX-90 dimer to the cardiolipin chains can prevent the shifting apart of the cardiolipin chains altered by oxidation, improve the bilayer thickness of mitochondrial membranes and prevent the release of MT-DNA.

Accordingly, the three mechanisms by which NDX-90 can be prophylactic and therapeutic are as follows:—
1) Firstly, the reduction of glucagon levels to normality reduces ACE2 levels sufficiently to prophylactically prevent Covid-19 infection, or therapeutically prevent disease progression and death.
2) Secondly, glucagon and glutamate are stoichiometrically co-secreted, and therefore, reduced glucagon secretion is stoichiometrically accompanied by reduced glutamate secretion. Glutamate promotes viral multiplication and glutamate excitotoxicity is the cause of neurological complications in Covid-19 disease. Therefore, by reducing both glucagon and glutamate secretion, NDX-90 can be highly effective in preventing or ameliorating coronavirus infection.
3) Thirdly, mitochondrial DNA and cardiolipin leakage from damaged mitochondria observed in severe Covid-19 disease, is inflammatory and results in acute respiratory distress syndrome (ARDS), cytokine storm, and multi-organ failure. NDX-90 dimer binds to cardiolipin, which is also dimeric and stabilises damaged cardiolipin-rich mitochondrial membranes. NDX-90, which is cell penetrating, has already shown a highly significant therapeutic effect in Type 2 diabetes which is a disease associated with mitochondrial dysfunction and subject to severe Covid-19 infection outcomes.

Accordingly, NDX-90, a comprehensive fast-acting treatment, addresses primary infection levels (glucagon and ACE2) and also disease aggravating factors (high glutamate) involved in viral proliferation, neurotoxicity, and mitochondrial destabilisation, which releases pro-inflammatory mitochondrial DNA and cardiolipin. Therefore, the NDX-90 peptide can be used in the effective prophylaxis, amelioration, or treatment of a coronavirus infection and symptoms in an infected subject.

### REFERENCES

1. CDC COVID-19 Response Team. Preliminary Estimates of the Prevalence of Selected Underlying Health Conditions Among Patients with Coronavirus Disease 2019-United States, Feb. 12-Mar. 28, 2020. MWMR, Apr. 3, 2020, Vol 69, No 13.
2. Ramchand J., Patel S. K., et al. Elevated plasma angiotensin converting enzyme 2 activity is an independent predictor of major cardiac events in patients with obstructive coronary artery disease. PLOS ONE journal.pone.0198144 Jun. 19, 2018.
3. Soro-Paavonen A, Gordin D, Forsblom C et al. Circulating ACE2 activity is increased with type 1 diabetes and vascular complications. J Hypertens 30: 375-383, 2012.
4. Anguiano L, Riera M, Pascual J et. al. Circulating angiotensin-converting enzyme 2 activity in patients with chronic kidney disease without previous history of cardiovascular disease. Nephrol Dial Transplant 30:1176-1185, 2015.
5. Gilbert A., Liu J., Cheng G., et al. A review of urine angiotensin converting enzyme 2 in diabetes and diabetic nephropathy. Biochem Med (Zagreb) 2019; 29(1): 010501.
6. Yang J K, Feng Y, Yuan M Y et. al. Plasma glucose levels and diabetes are independent predictors for mortality and morbidity in patients with SARS. Diabetic Medicine, 23 (6): 623-628, 2006.
7. Jia H P, Look D C, Shi L, Hickey M et. al. ACE2 receptor expression and Severe Acute Respiratory syndrome Coronavirus Infection depend on differentiation of human airway epithelia. Journal of Virology, 79:14614-14621, 2005.
8. Qiao Q, Jousilahti P, Eriksson J et. al, Predictive properties of Impaired Glucose Tolerance for Cardiovascular risk are not explained by the development of overt diabetes during follow-up. Diabetes Care, 26: 2910-2914, 2003.
9. Sechi L A, Catena C, Zingaro L, et. al. Abnormalities of glucose metabolism in patients with early renal failure. Diabetes, 51:1226-1232, 2002.
10. Nathan D M, Davidson M B, DeFronzo R A et. al. Impaired fasting glucose and impaired glucose tolerance. Diabetes Care, 30:753-759, 2007.
11. Abdul-Ghani M A, Tripathy D, De Fronzo R A. Contribution of β-cell dysfunction and insulin resistance to the pathogenesis of impaired glucose tolerance and impaired fasting glucose. Diabetes Care, 29:1130-1139, 2006.
12. Faerch K, Vistsen D, Pacini G et. al. Insulin resistance is accompanied by increased fasting glucagon and delayed glucagon suppression in individuals with normal and impaired glucose regulation. Diabetes, 65: 3473-3481, 2016.
13. Ichikawa R, Takano K, Fujimoto K, et. al. Basal glucagon hypersecretion and response to oral glucose load in prediabetes and mild type 2 diabetes. Endocrine Journal, 66:663-675, 2019.
14. Reaven G M, Chen Y D, Golay A, et. al. Documentation of hyperglucagonemia throughout the day in nonobese and obese patients with noninsulin-dependent diabetes mellitus. J Clin. Endocrinol. Metab. 64:106-110, 1987.
15. Estall J L, Drucker D J. Glucagon and Glucagon-Like Peptide Receptors as Drug Targets. Current Pharmaceutical Design 2006; 12: 1731-1750
16. Huypens P, Ling Z, Pipeleers D, Schuit F. Glucagon receptors on human islet cells contribute to glucose competence of insulin release. Diabetologia 2000; 43:1012-1019
17. Ahren B. Glucagon secretion in relation to insulin sensitivity in healthy subjects. Diabetologia 2006 January; 49(1):117-22. Epub 2005 Dec. 17
18. Liu J, Li X, Lu Q et al. AMPK: a balancer of the renin-angiotensin system. Bioscience Reports, 2019, 39 BSR 20181994.
19. Hardie, DG. Minireview: the AMPK-activated protein kinase cascade: the key sensor of cellular energy status. Endocrinology, 2003; 144:5179-5183.
20. Longuet C, Sinclair E M, Maida A et. al. The glucagon receptor is required for the adaptive metabolic response to fasting. Cell Metabolism 8:359-371, 2008.
21. Hardie, DG. AMP-activated protein kinase: a key regulator of energy balance with many roles in human disease. J. Intern. Med., 2014; 276: 543-559.
22. Meng R S, Pei Z H et al. Adenosine-monophosphate-activated protein kinase inhibits cardiac hypertrophy through reactivating peroxisome-activated receptor-alpha signaling pathway. Eur. J. Pharmacol., 2009; 620: 63-70.
23. Li X., Liu J. et al., AMPK: a therapeutic target of heart failure—not only metabolism regulation. 2019; Biosci. Rep. 39, BSR20181767.
24. Kim S G., Kim J R. et al., Quercetin-induced AMP-activated protein kinase activation attenuates vasoconstriction through LKB1-AMPK signaling pathway. J. Med. Food, 2018; 21:146-153.
25. Gao F., Chen J. et al. A potential strategy for treating atherosclerosis: improving endothelial function via AMP-activated protein kinase. Sci. China Life Sci., 2018; 61: 1024-1029.
26. Hivashi M, Yamada H et al. Secretory Granule-mediated Co-secretion of L-Glutamate and Glucagon Triggers Glutamatergic Signal Transmission in Islets of Langerhans, JBC.2003; 278:1966-1974.
27. Cabrera O. Jacques-Silva M C et al. Glutamate Is a Positive Autocrine Signal for Glucagon Release, Cell Metabolism, 2008; 7:1-10.
28. Ceriello A, Standl E, et al. Issues of Cardiovascular Risk Management in People With Diabetes in The Covid-19 Era. Diabetes Care, 2020; 43(7): 1427-1432.
29. Scozzi D, Cano M et al. Circulating mitochondrial DNA is an early indicator of severe illness and mortality from COVID-19. JCI Insight, 2021:6(4):e143299 https://doi.org/10/1172/jci.insight.143299
30. Paez-Franco J C, Torres-Ruiz J et al, Metabolomics analysis reveals a modified amino acid metabolism that correlates with altered oxygen homeostasis in COVID-19 patients, Nature Scientific Reports, 2021; 11:6350.
31. Chen T, Zang X, Long Y, Yu H, Ran X, Gao Y, Lu H, Xie X, Chen X, et al. The association of plasma free amino acids with liver enzymes in type 2 diabetes patients. J Endocrinol Invest. 2012, 35:772-775.
32. Krishnan S, Nordqvist H et al. Implications of central carbon metabolism in SARS-Cov-2 replication and disease severity. bioRxiv, 2021; doi:https://doi.org/10.1101/2021.02.24.432759
33. Engin A B, Engin E D et al. Current opinion in neurological manifestations of SARA-CoV-2 infection. Current Opinion in Toxicology, 2021; 25: 49-56.
34. Romero-Sanchez C M, Diaz-Maroto I et al. Neurologic manifestations in hos;ilazed patients with COVID-19: the ALBACOVID registry. Neurology, 2020; 95: e1060-e1070.
35. Zeng Y., Hu F B et al. Metabolites of Glutamate Metabolism Are Associated With Incident Cardiovascular Events in the PREDIMED PREvencion con Dleta MEDiterranea (PREDIMED) Trial. J Am Heart Assoc. 2016; 5:e003755.
36. Paradies G, Paradies V, De Benedictis V et al. Functional role of cardiolipin in mitochondrial bioenergetics. Biochimica and Biophysica Acta, 2014; 1837: 408-417.
37. Vahaheikkila M, Peltomaa T, Rog T et al. How cardiolipin peroxidation alters the properties of the inner mitochondrial membrane. Chemistry and Physics of Lipids, 2018; 214: 15-23.
38. Kragstrup T W, Sing H S et al, Plasma ACE2 predicts outcome of COVID-19 in hospitalized patients PLOS ONE, 2021; 16(6):e0252799.

1-26. (canceled)

27. A method of treating, preventing or ameliorating a coronavirus infection or symptoms in an infected subject, the method comprising, administering, or having administered, to a subject in need of such treatment, a therapeutically effective amount of a peptide or a nucleic acid encoding a peptide,
wherein the peptide comprises i) an amino acid sequence substantially as set out in SEQ ID No: 1, or ii) an amino acid sequence that has at least 80% sequence identity to SEQ ID No: 1.

28. The method according to claim 27, wherein the peptide is linked together with a second peptide to form a dimer,
wherein the second peptide comprises i) an amino acid sequence substantially as set out in SEQ ID No: 1, or ii) an amino acid sequence that has at least 80% sequence identity to SEQ ID No:1.

29. The method of claim 28, wherein the peptides are linked by a cysteine residue at the N-terminal of each peptide.

30. The method according to claim 27, wherein the peptide or the nucleic acid, is capable of reducing and/or inhibiting the production of anti-anti-T cell receptor antibodies in a subject compared to the level of anti-anti-T cell receptor antibodies in an untreated subject.

31. The method according to claim 27, wherein the peptide, or the nucleic acid, is capable of reducing and/or inhibiting glucagon secretion in a subject compared to the level of glucagon secretion in an untreated subject, optionally wherein glucagon secretion is reduced and/or inhibited in the subject's pancreatic islets compared to the level of glucagon secretion in an untreated subject.

32. The method according to claim 27, wherein the peptide or the nucleic acid, is capable of reducing and/or inhibiting glutamate secretion in a subject compared to the level of glutamate secretion in an untreated subject, and/or wherein the peptide, derivative or analogue thereof, or the nucleic acid, is capable of reducing and/or inhibiting ACE2 overexpression in a subject compared to the level of ACE2 expression in an untreated subject.

33. The method according to claim 27, wherein the peptide or the nucleic acid, is capable of binding to cardiolipin, optionally wherein the peptide, derivative or analogue thereof, or the nucleic acid, is capable of reducing and/or inhibiting the release of mitochondrial DNA (mtDNA) from the mitochondria in a subject, compared to the level of mtDNA release from the mitochondria in an untreated subject.

34. The method according to claim 27, wherein the coronavirus is a virus belonging to the coronavirus group of disease-causing pathogens, which targets the Angiotensin Converting Enzyme 2 (ACE2) receptor on a host cell for infection thereof.

35. The method according to claim 27, wherein the coronavirus is selected from MERS, SARS-CoV-1 and SARS-CoV-2, and/or wherein the coronavirus is SARS-CoV-2.

36. The method according to claim 27, wherein the peptide or the nucleic acid is capable of treating, preventing or ameliorating a coronavirus infection or symptoms in an infected subject who suffers from an underlying disease, wherein the disease is associated with impaired glucose tolerance or elevated fasting plasma glucose, optionally wherein the disease is associated with high glucagon levels or insulin resistance.

37. The method according to claim 36, wherein the subject is glucose-intolerant.

38. The method according to claim 36, wherein the disease is selected from a group consisting of: diabetes; Type 1 diabetes; Type 2 diabetes; hypertension; cardiovascular disease; kidney disease; and lung disease, optionally wherein the subject is a SARS-CoV-2-infected diabetic patient.

39. The method according to claim 27, wherein the subject does not suffer from an underlying disease, optionally wherein the subject is a SARS-CoV-2-infected non-diabetic patient.

40. The method according to claim 27, wherein the peptide or the nucleic acid is capable of treating, preventing or ameliorating a coronavirus infection or symptoms in an infected subject who is in their 20's, 30's, 40's, 50's, 60's, 70's, 80's or 90's.

41. The method according to claim 27, wherein the nucleic acid comprises a nucleotide sequence substantially as set out in SEQ ID No:2 or 3, and/or wherein the nucleic acid encoding the peptide, derivative or analogue thereof is a genetic construct, and/or wherein the nucleic acid molecule or genetic construct is provided in a recombinant vector.

42. A method of treating, preventing or ameliorating hyperglucagonemia, hyperinsulinemia and/or a condition characterized by high or excessive glutamate in a subject, the method comprising, administering, or having administered, to a subject in need of such treatment, a therapeutically effective amount of a peptide or a nucleic acid encoding a peptide,
wherein the peptide comprises i) an amino acid sequence substantially as set out in SEQ ID No: 1, or ii) an amino acid sequence that has at least 80% sequence identity to SEQ ID No: 1.

43. The method according to claim 42, wherein the condition is Type 2 or Type 1 diabetes, hypertension, chronic heart disease, cardiovascular disease, kidney disease, chronic lung disease, obesity, cancer, Impaired Glucose Tolerance (IGT), Impaired Fasting Glucose/Glucagon (IFG), Alzheimer's disease, chronic schizophrenia, Major Depressive Disorder (MDD), Autism Spectrum Disorder, Multiple Sclerosis, Parkinson's disease or neuro-muscular degenerative disorder.

44. The method according to claim 42, wherein the peptid2 or the nucleic acid is used in the effective prophylaxis, amelioration, or treatment of a coronavirus infection or symptoms in an infected subject, preferably by reducing and/or inhibiting ACE2 expression in a subject compared to the level of ACE2 expression in an untreated subject.
